(19) United States
(12) Patent Application Publication
(10) Pub. No.: US 2017/0247720 A1
(43) Pub. Date: Aug. 31, 2017

(54) BIOLOGICAL PROCESS FOR PRODUCING MAGNETIC NANOPARTICLES

(71) Applicant: Cristhian Rene LOPEZ NAVARRO, Guanajuato (MX)

(72) Inventors: Cristhian Rene LOPEZ NAVARRO, Guanajuato (MX); Willie Ossiel OCANA RANGEL, Guanajuato (MX); Diana RAMIREZ SAENZ, Guanajuato (MX)

(73) Assignee: Cristhian Rene LOPEZ NAVARRO, Guanajuato (MX)

(21) Appl. No.: 15/521,649

(22) PCT Filed: Nov. 10, 2014
(86) PCT No.: PCT/MX2014/000177

§ 371 Date: Apr. 25, 2017

(51) Int. Cl. C12P 3/00

(52) U.S. Cl. CPC C12P 3/00

(57) ABSTRACT

A process for obtaining gold magnetic nanoparticles or of other metallic elements by fermenting a culture medium in the presence of producer microorganisms such as Brevibacterium halotolerans, Bacillus mojavensis, Bacillus subtilis subsp. Inaquosorum, Bacillus subtilis subsp. Spizizenii, Bacillus tequilensis, Bacillus amyloliquefaciens subsp. Amyloliquefaciens, Bacillus siamenensis, Bacillus amyloliquefaciens subsp. Plantarum, Bacillus subtilis, Bacillus subtilis subsp. Inaquosorum, Bacillus atrophaeus, or Bacillus vallismortis, inter alia. The process of the invention can be used to effectively control the size and shape of the nanostructure to be obtained, with production levels above those found at laboratory level.

### CROSS REFERENCE TO RELATED APPLICATION

This application is a national stage entry of PCT/MX2014/000177 filed Nov. 10, 2014, under the International Convention.

### FIELD OF THE INVENTION

The present invention is related to the fields of nanotechnology and biotechnology as it refers to a biological process for the production of nanostructures from biotechnological processes using producer microorganisms, preferable from bacteria of the genus Brevibaterium haloterans and/or Bacillus mojavensis, from which metallic magnetic nanoparticles are obtained, preferably gold magnetic nanoparticles. Particularly, the present invention is related to biotechnological processes susceptible of being executed to obtain industrial levels of magnetic nanoparticles in quantities and qualities equivalent to or above those usually obtained at laboratory level, and which are suitable to be used in various fields of industry such as in nanomedicine, drug delivery and other uses. The present invention is considered that is related to the field of nanobiotechnology or bionanotechnology.

### BACKGROUND OF THE INVENTION

Nanotechnology is the ability to manipulate matter in nanometres for creating structures with a different molecular organization. The term “nanoparticle” or “nanomaterial” is used to refer to particles which have a diameter of between 1 and 100 nm. The term “nanodispersion” is used to identify particles with a uniform size in a disperse phase. In process engineering, “biomass” makes reference to cells, in this case of the microorganism being used as a biocatalyst.

Nanoparticles have gained great interest because of the particularities of their optical, magnetic, electric and catalytic properties, which make them useful in various applications in the areas of nanotechnology, catalysis, non-lineal optics, electronics, labelling and drug release and biomedicine. Many of these properties and their possible applications are strongly influenced by the size and the shape thereof: spheres, bars, discs, prisms, etc. That is why during the last years different physical and chemical methods of nanoparticles preparation have been developed with the tendency to control the morphological features of the obtained product.

The chemical methods for the production of nanoparticles include chloride and citrate reduction methods for the preparation of gold colloidal nanoparticles, while the physical methods include vapour deposition, lithographic processes or metal ion reduction by radiolysis.

However, the methods of the above-mentioned techniques have various disadvantages: the chemical methods are dangerous for the environment as in some cases specific levelling agents such as acids are used with the purpose of restricting the size of metal particles, and so, at the end of the process there is waste toxic for the environment.

Nanobiotechnology is currently considered as the confluence between nanotechnology and biotechnology. It includes all the methods and techniques up to nanoscale (10⁻⁹ m), which could serve to control and characterize biological matter. These techniques are currently used to study and modify biomolecules and cells. Apart from the application to study biosystems, the researchers also learn from the biology to create new devices up to micronanoscale and to understand the processes related to life at nanometric scale. The nanotechnology applied to health (usually termed nanomedicine) is one of the key technologies to achieve more precise early diagnostic systems (the earlier the diagnosis, the more options and higher efficiency of treatments), treatments more directed to therapeutic targets (which reduce side effects) and improved therapy follow up (Samitier I Martí, J. (2014). Nanobiotecnology i nanomedicina. Trebells de la Societat Catalana de Biologíc, 131-138).

Within the aspects included in the nanobiotechnology, it is the ability thereof to manipulate matter to nanometres to create structures with a different molecular organization which, when using the biotechnological methods and processes, new materials are created and obtained and new products and processes are developed based on the increase on the ability to see and manipulate the atoms and the molecules (Pina, K. V., Pinto, L. R., Moratori, R. B., de Souza, C. G., & Barbastefano, R. G. (2006). Nanotecnologia e nanobiotecnologia: estado da arte, perspectivas de inovaçao e investimentos. Revista Gestao Industrial, 2(2)).

It has been reported that some species of microorganisms are capable of reducing Au⁺³ to Au⁰ ions, producing octahedrons within cells walls or sulphate reducing heterotrophic bacteria to destabilize a gold thiosulfate complex (I) to elemental gold, intracellularly; on the other hand, microorganisms capable of reducing Au III to metallic gold by the action of the gold reductase enzyme in the presence of hydrogen as electron donors, have also been reported.

Taking advantage of this feature, recently in the field of nanobiotechnology, different biological methods for the synthesis of gold nanoparticles and other metals have emerged which, in spite of being novel processes and showing advantages with respect to physical or chemical methods, they have not been able to compete industrially against them because their development and scaling is still limited by certain factors such as low yields, high costs of the culture medium, long production terms, costly and inefficient purification methods, etc. Among some reported work the following are worth mentioning:

The patent ES 2379 017, Method for producing silver or gold nanoparticles, describes a method for producing silver or gold nanometric-sized particles by a biological process based on probiotic bacteria of the genus Lactobacillus under specific conditions for the production of silver or gold nanoparticles with the purpose of improving their microbial efficacy. It refers to a way of obtaining a silver nanoparticles solution to be used in antimicrobial compositions, which, even when the obtained silver nanostructures could be recovered, the yields tend to be very low and are only suitable to be applied in this type of antimicrobial compositions.

The publication WO 2013/143017, Use of Botrytis cinerea for the production of gold nanoparticles, describes the synthesis of gold nanoparticles using the microbial strain, its spores, its hyphae, mycelium, sclerocytes, and intracellular and extracellular organic molecules (such as proteins, nucleic acids, polysaccharides, lipids and secondary metabolites). Even when it is revealed that gold nanoparticles are obtained, the description of this patent document discloses proportions and conditions that are carried out in a laboratory, but could hardly be escalated to costs and yields considered as industrial.

Patent document JP201312403, Microorganism forming gold nanoparticles and method of nanoparticles formation using the same, discloses a method for the formation of gold nanoparticles using a modified strain of the genus Collimonas. This invention describes steps and procedures, which are carried out at the test tube and Erlenmeyer flask level under controlled laboratory conditions which, like in the above-mentioned procedures would not result efficient at all if escalated.

The patent publication US 2012/108425, Biosynthesis of gold and silver nanoparticles for stability and extended shelf-life of antagonistic activities, mentions a method for obtaining organisms of the genus Pseudomonas auroginosa, Trichoderma atroviride and Streptomyces spp. capable of synthesizing gold as well as silver nanoparticles with the purpose of employing these particles for stabilizing the antimicrobial properties of biocontrol agents, specially for fighting the effects caused by the microorganism Cercospora theae. In this case a gold and silver nanoparticles concentration capable of being used in the formulation of biocontrol agents is obtained, and so, it is of no interest to recover said gold and silver nanoparticles, but rather concentrations thereof to be used in the formulation of compositions.

The patent publication US 2002/0174743 A1, Process for the preparation of nanometric colloidal metal nanoparticles, discloses the use of the microorganism Fusarium oxysporum. Similarly to the above-mentioned patent applications, this invention is related to obtaining colloidal gold nanoparticles to be used in the biological control of microorganisms and does not intend to recover or separate the nanostructures for other uses in nanotechnology.

Document DE1020006001759, Synthesis of metallic nanoparticles and nanocrystals particularly useful for silver disinfection, discloses the use of the microorganism Lubomirskia baicalensis for the production of gold nanoparticles.

The patent publication US 2009/0239280 A1, Method for producing metallic nanoparticles, mentions that metallic nanoparticles can be produced with different compositions, be it silver, gold, zinc, mercury, copper, palladium, platinum or bismuth using Lactobacillus fermentum bacteria.

Document US 2011/0124131 A1, Method for preparing metallic nanoparticles using a metal binding protein, describes the use of metal binding proteins obtained from various model organisms, such as Homo sapiens, Arabidopsis thaliana, Mus musculus, Saccharomyces cerevisiae, etc., to produce metallic nanoparticles, but transforming a coding gene to express a metallic protein in ionic medium so that metallic structures are produced in the microorganisms.

The patent publication US 2010/0055199 A1, Synthesis of nanoparticles by fungi, describes the production of silver nanoparticles using fungi of the Trichoderma reesei species, in a manner so as to produce an enzymatic catalyser for reducing silver salt in nanoparticles.

Solutions such as those above-referenced in the patent documents and other documents which report scientific research results, allow us to demonstrate that there are no true technical proposals which allow the obtaining of magnetic nanoparticles using producer microorganisms with the quality, amount and features which could represent true alternatives of industrial application. It is not sufficient to carry out laboratory processes with relative results obtaining the intended nanomaterials, but rather, the used processes do not consider their capacity to be implemented to substitute the current chemical and physical processes, in the quantity and cost of the products and above all the quality and features only obtained with biotechnological processes.

For example, the method reported by Lengke et al., comprises a method for the production of gold nanoparticles by the Plectonema borynum microorganism. However, the synthesis was carried out during a period of 1 month (Lengke, M. F., Fleet, M. E., & Southam, G. (2006). Morphology of gold nanoparticles synthesized by filamentous cyanobacteria from gold (I)-thiosulfate and gold (III)-chloride complexes. Langmuir, 22(6), 2780-2787), which would evidently represent not only serious economic difficulties to obtain gold nanoparticles, but the time and scale of manufacturing would definitely not make it viable at all to obtain gold nanoparticles for applications in the industry, as it would not be a cost-effective process.

Another case is the work published by Govindaraju et al., which highlights that the production of gold nanoparticles requires of a reaction time of about 120 hours obtaining nanoparticles of between 6-10 nm (Govindaraju, K., Basha, S. K., Kumar, V., G., & Singaravelu, G. (2008). Silver, gold and bimetallic nanoparticles production using single-cell protein (Spirulina platensis) Geitler. Journal of Materials Science, 43(15), 5115-5122); however, it does not mention or there is no reference to the obtained conversion rate nor whether or not it is possible to scale up the used system.

Another work is that carried out and reported by Feng et al., which mentions that the maximum yield obtained for gold biosorption was 92.43 mg of HACL₄/g of dry biomass obtaining nanoparticles of between 10-20 nm (Feng, Y., Yu, Y., Wang, Y., & Lin, X. (2007). Biosorption and bioreduction of trivalent aurum by photosynthetic bacteria Rhodobacter capsulatus. Current Microbiology, 55(5), 402-408). Evidently, these performance quantities and parameters are far from reaching a commercial level of production scale.

In the article reported by Du et al., a method is reproduced for the production of gold nanoparticles in a period of 120 hours obtaining 20 to 30 nm nanoparticles (Du, L., Jiang, H., Liu, X., & Wang, E. (2007). Biosynthesis of gold nanoparticles assisted by Escherichia coli DH5a and its application on direct electrochemistry of haemoglobin. Electrochemistry Communications, 9(5), 1165-1170); however, there is no mention of the conversion rate or the possible scaling up of this process. What is deduced is only a proposal carried out at laboratory level with a poorly attractive performance for industrial production scales and commercial requirements.

The article by Husseiny et al., contemplates the extracellular production of gold nanoparticles using a method similar to those used in the above-mentioned prior art, obtaining 40 nm-sized nanoparticles (Husseiny, M. I., El-Aziz, M. A., Badr, Y., & Mahmoud, M. A. (2007). Biosynthesis of gold nanoparticles using Pseudomonas aeruginosa. Spectrochimica Acta Part A: Molecular and Biomolecular Spectroscopy, 67(3), 1003-1006). Even when it is directed to obtaining gold nanoparticles, there is no mention of a specific method of separation of the obtained nanostructures or of the possibility for scaling up the process from the elements here considered.

The work reported by Suresh et al., uses a similar method for producing gold nanoparticles extracellularly. This reference is the only one, which mentions a yield for obtaining nanoparticles of 174 mg per litre of preparation (Suresh, A. K., Pelletier, D. A., Wang, W., Broich, M. L., Moon, J. W., Gu, B., . . . & Doktycz, M. J. (2011). Biofabrication of discrete spherical gold nanoparticles using the metal-reducing bacterium Shewanella oneidensis. Acta biomaterialia, 7(5), 2148-2152). However, it does not contemplate the production of these nanoparticles in a reactor or in a production at the industrial level.

The article by Nangia et al., uses a Stenotrophomonas maltophilia strain for extracellularly synthesizing the gold nanoparticles obtaining them with an average size of 40 nm, wherein the authors present the possible synthesis mechanism for this species in particular (Nangia, Y., Wangoo, N., Sharma, S., Wu, J. S., Dravid, V., Shekhawat, G. S., & Suri, C. R. (2009). Facile biosynthesis of phosphate capped gold nanoparticles by a bacterial isolate Stenotrophomonas maltophilia. Applied Physics Letters, 94(23), 233901). However, because in this reference there is no mention of the system or purification step and the scope of the process up to superior scales, it is easily assumed that this processes does not go beyond the laboratory level.

In the case of the biosynthesis process for gold nanoparticles, Cai et al., uses a Magnetospirillum gryphiswaldense MSR-1 strain for the reduction of gold ions to metal with zero valence in aqueous medium, obtaining spherical nanoparticles with diameters of between 12-40 nm and verifying the effect of the initial molar concentration and the pH (Cai, F., Li, J., Sun, J., & Ji, Y. (2011). Biosynthesis of gold nanoparticles by biosorption using Magnetospirillum gryphiswaldense MSR-1. Chemical Engineering Journal, 175, 70-75). However, even though it mentions spherical nanoparticles, the authors do not have a clear control of the synthesis of nanoparticles with respect to shape, but rather the sizes thereof; also, as like the other above-mentioned articles, judging by the scope thereof it is considered that this only reaches laboratory levels.

In another published work case, Castro-Longoria et al., uses the same technique for the production gold, silver and biometallic nanoparticles (Castro-Longoria, E., Vilchis-Nestor, A. R., & Avalos-Borja, M. (2011). Biosynthesis of silver, gold and bimetallic nanoparticles using the filamentous fungus Neurospora crassa. Colloids and Surfaces B: Biointerfaces, 83(1), 42-48). However, the method for culturing conidia would present technical complications to carry it out at an industrial level, since, in this reference the authors used the Vogels Minimal Medium and is carried out in a solid system, as the yields for an industrial level would be reduced as a culture in liquid would have to be carried out and with a more cost-effective medium.

Finally, in the case of Konishi et al., the anaerobic bacterium Shewanella algae was used in a method in which gold nanoparticles were produced (Konishi, Y., Tsukiyama, T., Ohno, K., Saitoh, N., Nomura, T., & Nagamine, S. (2006). Intracellular recovery of gold by microbial reduction of AuCl⁻₄ ions using the anaerobic bacterium Shewanella algae. Hydrometallurgy, 81(1), 24-29). However in this method it is not possible to control the shape or size thereof; also the biosynthesis must be carried out in anaerobiosis, which represents a technical disadvantage to carry it out at an industrial level since the biomass production periods would be increased; on the other hand the nanoparticles synthesized by this bacteria are localized in the peri plasma, which a relatively small region of the cell, reason for which the yields, which are not mentioned in this reference, would be decreased unlike if they were produced in the plasma or extracellularly.

It can be appreciated from this review of the state of the prior art that the reported processes do not contribute with sufficient elements to carry them out at industrial levels, even in chemical and traditional processes there are serious limitations to achieve yields and capacities which would result industrially attractive, especially for the handling of materials, the separation processes, rates of contaminants of the processes themselves, and the shape of the obtained nanostructures.

The state of the prior art comprises the order of more than 100 patents and more than 1,760 paper articles for the production and use of gold nanoparticles by biological methods with a variety of microorganisms from different types but with similar processes. Practically all of them have focused on the initial part of demonstrating the concept of obtaining magnetic nanoparticles in any shape from any type of producer microorganism, by the steps which guarantee the medium so that said microorganisms produce the nanoparticles from precursor materials and culture medium, thereby obtaining a biomass which suggests the presence of magnetic nanoparticles independently of the cost, time, quantity and qualities of the obtained nanoparticles and the viability to widen the scopes of these methods to processes of industrial level.

It can be asserted that neither of the proposals in the state of the prior art contribute with the elements of knowledge necessary to resolve the technical problem which involves separating, characterizing and obtaining nanoparticles under conditions which allow their use afterwards to be functionalized and applicable, for example in nanomedicine. In fact, it is considered that the progress state of the art up to the time of the present invention does not allow one to use the benefits of obtaining magnetic nanoparticles by biotechnological means in the potential applications, which exist because of the lack of availability thereof in the market under conditions of availability, quality, cost and flexibility which allow sustaining the development of their applications in an efficient and clear manner, reducing the risks of innovative concept.

With regards to the efficiency and capacity of the biotechnological processes, obtaining nanoparticles using biological means has a major complexity than the rest of the conventionally known and executable bioprocesses. The biotechnology has evolved from the knowledge generated in diverse subjects, both in the area of basic sciences as well as in engineering, particularly for the case of obtaining magnetic nanoparticles there is a special relevance not only the contribution of the knowledge elements from the basic sciences but the creative contributions in engineering.

Particularly, all of the cited references and others in the state of the prior art have systems for the biosynthesis at industrial level not higher than 1 Litre (and at times in volumes much lower than this) showing conversion levels of the precursor and also a control of the size and shape of the nanoparticle and a purification system which allows obtaining well established size ranges. All of this was achieved by the proposal of our process wherein selected producer microorganisms were used, prepared in culture medium and under control conditions which allow not only to obtain a biomass containing nanoparticles of interest but in such a shape which is possible to obtain amounts and qualities of nanoparticles not previously achieved until now with chemical traditional processes and even less with biotechnological processes.

The activities targeted to the scaling or scaling up of chemical processes represent the synthesis of the “know-How” built up during the different phases of the development process which go from experiments at laboratory level, the understanding of the chemical and physical processes which take place during the process, fluid-dynamic experiments, mathematic modelling, design and operation of pilot and industrial plants. The term “scaling” or “scale up” is usually defined as the design of a pilot or industrial plant capable of replicating the results obtained in the laboratory; however, this definition is limited, since experience has demonstrated that in reality there are no standard methods to follow during the innovation process. The current processes of industrial production are the result of assertive decisions, and in most cases, the consequences of errors. In the past, the decisions have not always been supported by experimental evidence, and even today, the operation of industrial plants is mostly based on experience. For the case of chemical technology, it can be said that a more certain definition of the “scaling up” term would be the mixture of “Know-How”, the innovative ideas, standard methodologies and intuitive criteria of the businessmen.

In the case of biotechnological processes the problem is even more complex, the scaling up. For example, in the case of hybridoma-based models, it has been referenced that to this date few animal cell cultures have been carried out up to scales superior to 10,000 L. Nevertheless, there is evidence from existing data that the behaviour to higher scales is deficient with respect to laboratory cultures. This is the result of both the scaling up procedure used as well as the impossibility to intensely stir and bubble up cultures because of the inherent fragility of the animal cells. The above can result in the formation of gradients in the dissolved oxygen tension (TOD). In this project a study on descending scaling as an option to know the effects that the presence of TOD gradients can have on the reactors at high scale on animal cells or hybridomas was carried out (“Estudio de Escalamiento Descendente del Proceso de Producción de Anticuerpos Monoclonales por Cultivo de Hibridomas”, José Antonio Serrato Pérez; Octavio Tonatiuh Ramírez Reivich. Departamento de Bioingeniería, Instituto de Biotecnología, Universidad Nacional Autónoma de México. Memorias del Congreso de la Sociedad Mexicana de Biotecnología y Bioingeniería 2001, Veracruz México).

Many of such problems in the case of hybridomas have been solved with time; however, in the case of biotechnological processes directed to obtaining nanostructures as is the case of the present invention, the technical challenges are much more complex than in the case of traditional chemical and biotechnological processes. In these cases where the pathways and scaling up conditions not only would respond in theory to reproduce the conditions of the processes carried out in laboratory, but to generate novel alternatives of process technology which have repercussions in the features of the nanostructured products.

That is how in the wake of increasing interest in the use of nanoparticles for the various technological and scientific applications, it is necessary to find a way of producing nanoparticles that is flexible and which is associated to low production costs and high efficiency and quality standards of the obtained materials.

About the previously exposed synthesis methods, those carried out in liquid phase in batch production generally present problems during the industrial production because of the need of big mixing tanks, wherein the stirring is less uniform than in the laboratory, generating localized areas of low pH and thermic differences, which trigger la precipitation of nanoparticles in a non-desired state (not magnetic, oxide/metal etc.) apart from the additional costs than in the solid-liquid separation processes, washing and drying; which require the synthesis in a moist pathway.

In the case of magnetite nanoparticles, few studies reported in the literature about the scaling up of nanoparticle synthesis, the synthesis methods in the gas phase such as spray pyrolysis, spray flame and CVD are presented as the most used ones, reaching a production of more than 90% in volume of nanoparticles in the last years (Wegner, K.; Pratsinis, S. E. Scale-up of nanoparticle synthesis in diffusion flame reactors. Chemical Engineering Science, 2003, 58, 4581-4589). That is why it is natural to think that these processes could be used for the production in mass of nanoparticles. Wegner et al., reported the production of up to 200 g/h of titanium oxide and silica nanoparticles by spray flame. In 1996, Kleijn et al., maintained that the design of a scaled up CVD reactor is mainly an empirical challenge based on trial and error methods, as the specific conditions of the process limit the applicability of the common rules of scaling up and design of reactors. All the above are complex chemical and physicochemical processes which have required actions directed to the optimization in the scaling up processes taking advantage of the learning and technical capabilities.

In the case of biotechnological processes, many of these actions in the scaling up cannot be left to experience actions and trial and error, especially when it is about the use of microorganisms for the biosynthesis of nanoparticles which must be obtained considering dimensional features and of quality which allow them to be used in different uses and applications as nanostructures of a given shape and size and above all, costs which allow their extended use in the fields of the industry.

It is because of the above that the applicant through the present invention, has appealed to a series of proposals in process and product technology under an inventive concept which efficiently allows biotechnological processes for obtaining nanostructures in amounts and qualities suitable from the cost efficiency and availability of materials perspectives for different uses, in which not only the biotechnological process itself is transcendent from the inventive point of view but rather it further comprises the selection, management and use of a type of microorganisms very particular which makes it possible to obtain nanoparticles in the volume and with features which are important for the use afterwards. It is because of the above that it is considered that the applicant in compliance with the present invention, has developed a novel model to carry out the biotechnological processes for the biosynthesis of metallic nanoparticles from precursor materials and producer microorganisms particularly selected and managed, under conditions which allow their application up to superior scales such as those observed in the prior art.

### BRIEF DESCRIPTION OF THE INVENTION

The present invention is related to a process for obtaining magnetic nanoparticles from the use of producer microorganisms and gold precursors at industrial levels, preferably of the Brevibacterium or Bacillus type, preferably from the Brevibacterium haloterans and/or Bacillus mojavensis, which not only comprises their obtaining in the biomass of the process containing the nanoparticles, but in the following steps of purification and obtaining of the nanostructures so that they are susceptible to be used because of their shape, size and features of high yield materials in diverse applications, among which are found the medical, pharmaceutical and diagnostic applications and others. This has been achieved by an inventive process which basically comprises and in function of the nanoparticles of interest and the producer microorganism of the Bacillus mojavensis type which has been carefully selected and characterised in a way so as to be able to handle the most important variables of the process, among which are found the temperature, pH, initial molar concentration of the precursor, aeration and stirring rate to obtain nanoparticles of particular sizes and with specific shapes and with yields which surpass the yields at a laboratory level and are considered to scales very close or considered industrial levels. One particularity of the process of the present invention is that by determined steps of the process it is possible to effectively control the size and shape of the nanoparticles which are obtained with which the critical variables of the process are controlled and the products are more suitable for their separation and purification, representing a highly efficient process which practically improves any other proposal of a biological method or process for obtaining similar nanoparticles.

It is therefore an object of the present invention to provide an improved process for the production of metallic nanoparticles using producer microorganisms and precursors of the corresponding metallic elements.

Another object of the present invention is to provide a biological process for the production of magnetic metallic nanoparticles, preferably gold and silver nanoparticles, among other metals, by which it is possible to effectively control the size and shape of the nanostructure to be obtained.

Another object of the present invention is to provide a biological process up to a scale superior to a laboratory so as to obtain gold magnetic nanoparticles or of other metallic elements from the use of producer microorganisms consisting of a bacteria selected from the group comprising: Brevibacterium halotolerans, Bacillus mojavensis, Bacillus subtilis subsp. Inaquosorum, Bacillus subtilis subsp. Spizizenii, Bacillus tequilensis, Bacillus amyloliquefaciens subsp. Amyloliquefaciens, Bacillus siamenensis, Bacillus amyloliquefaciens subsp. Plantarum, Bacillus subtilis, Bacillus subtilis subsp. Inaquosorum, Bacillus atrophaeus, or Bacillus vallismortis, amongst others.

Another object of the present invention is to provide a biological process at pilot and industrial scale for the production of gold nanoparticles and of other metallic elements, using preferably producer microorganisms of the type of bacteria of the genus Bacillus mojavensis.

A further object of the present invention is to provide a process by which gold nanoparticles can be obtained in liquid medium in reactors of the order of 7 litres for the intracellular synthesis of nanoparticles of preferred shapes and sizes.

Even another object of the present invention is to provide a process for obtaining gold nanoparticles and of other metallic elements, whose biomass can be efficiently processed to separate nanoparticles that by their size, shape and quality allow them to be used in biosensors, Raman spectroscopy, drug release, bioballistics, catalysers, components in integrated circuits, materials of high thermic conductivity, as part of a rail of immunological detection, amongst other uses.

This and other objects of the present invention will be better understood and will be observed with more detail throughout the following chapter and claims.

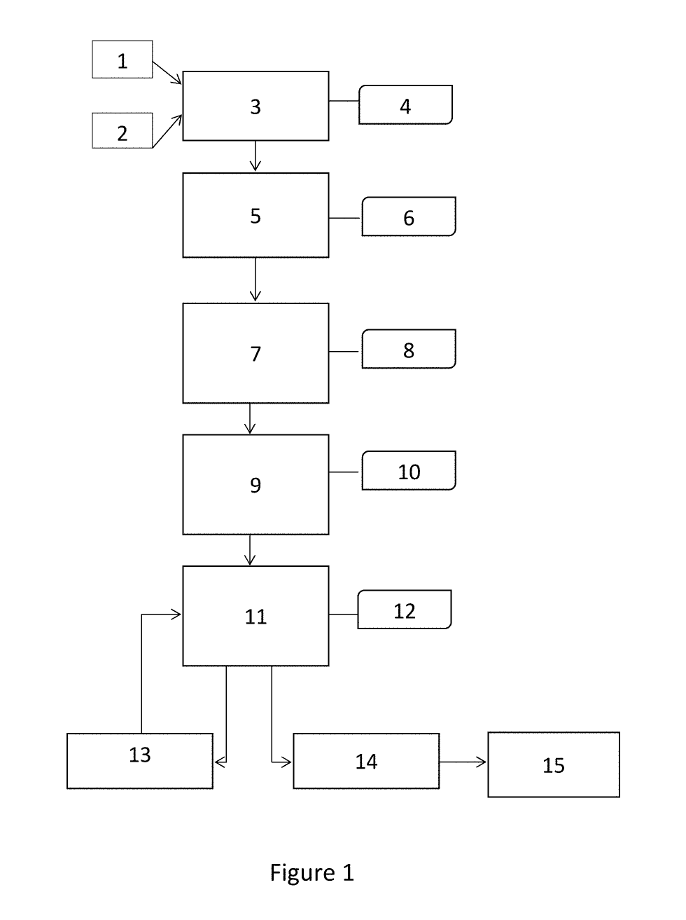

FIG. 1 shows a simplified block diagram of the object process of the present invention, which was developed under the synthesis methodologies of mixed bioprocesses, such as heuristic, algorithmic, and evolutionary adapted in particular for obtaining nanoparticles by biological methods at the industrial level.

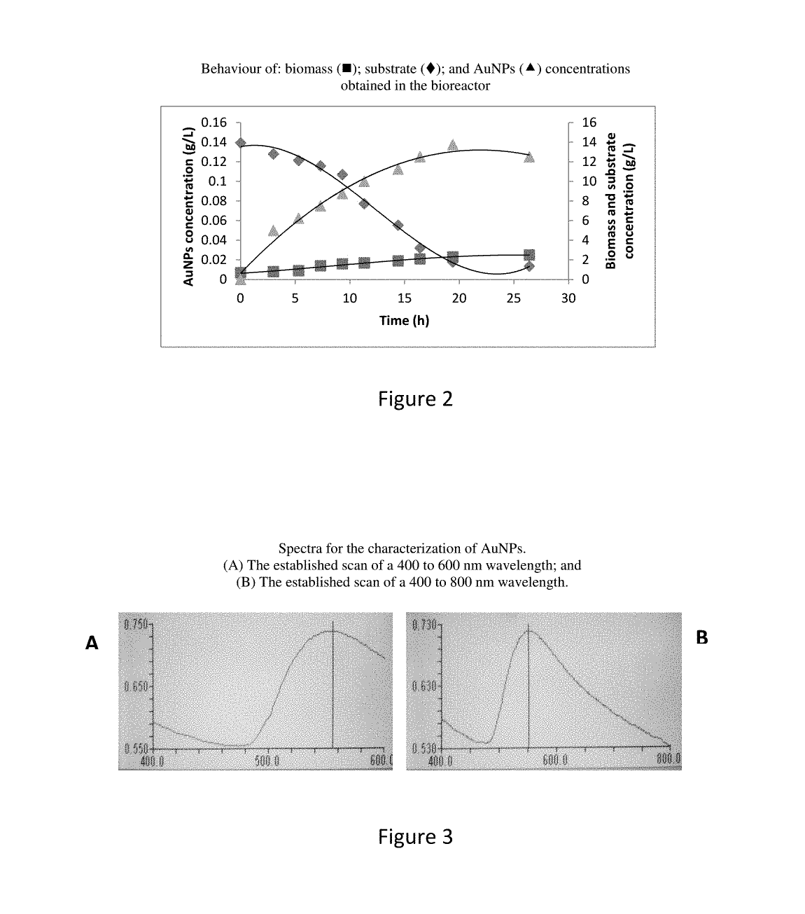

FIG. 2 shows a behaviour graph of the Bacillus mojavensis microorganism, which was part of the analysis of the kinetic parameters such as specific growth rate of the microorganism, biomass yield/substrate, biomass/product and substrate/product. Briefly, the obtained gold nanoparticles concentrations with respect to the biomass and substrate concentrations are observed according to the description for Example 2 of the present invention.

FIG. 3 shows images of some plasmon characteristic of nanoparticles solutions of an average size of 20 nm, useful for the characterization of the gold nanoparticles obtained according to the present invention.

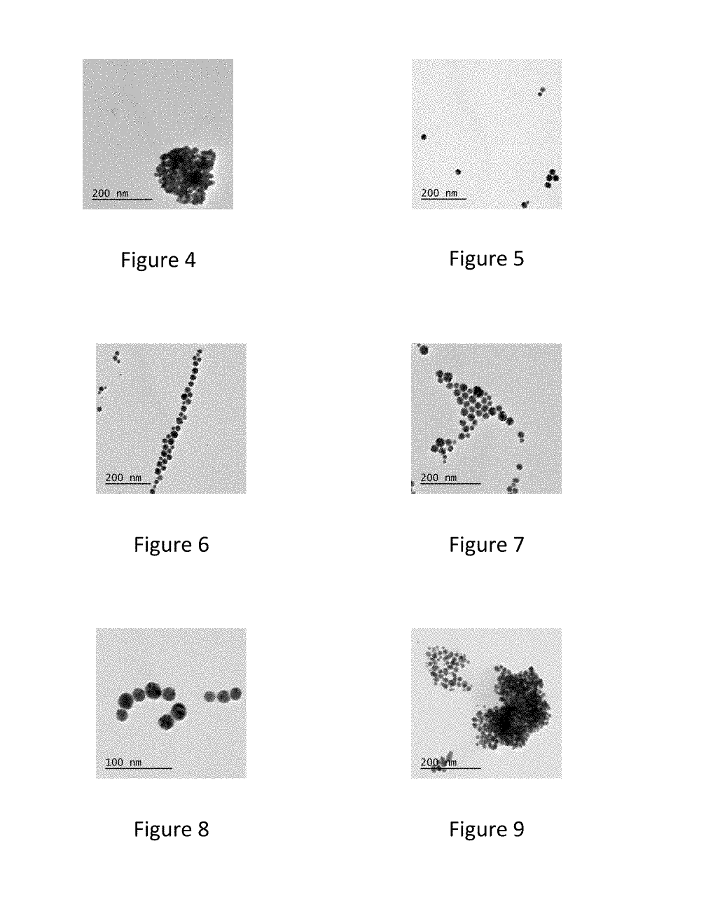

FIGS. 4 to 9 show microphotographs obtained by TEM from the obtained nanoparticles according to the present invention. In said microphotographs the spherical nanoparticles can be appreciated, mono dispersed with a size distribution of 20 nm, corresponding to samples obtained from the solution to which the UV-vis from FIG. 3 was carried out.

### DETAILED DESCRIPTION OF THE INVENTION

The present invention is related to a process for the production of nanostructures from biotechnological methods using selected or genetically modified producer microorganisms, in which there is particular reference to the production of gold magnetic nanoparticles, preferably crystals with a particle size of between 1 and 100 nm from microorganisms selected preferably from the group comprising the following types of bacteria: Brevibacterium halotolerans, Bacillus mojavensis, Bacillus subtilis subsp. Inaquosorum, Bacillus subtilis subsp. Spizizenii, Bacillus tequilensis, Bacillus amyloliquefaciens subsp. Amyloliquefaciens, Bacillus siamenensis, Bacillus amyloliquefaciens subsp. Plantarum, Bacillus subtilis, Bacillus subtilis subsp. Inaquosorum, Bacillus atrophaeus, or Bacillus vallismortis.

It is considered that the success of the process and the inventive merit of the present invention for obtaining gold nanoparticles with a yield up to scales superior to laboratory level not only because of the quantity but the quality of the obtained nanoparticles, must not be considered that they solely depend on scaling the bioprocess in a conventional manner, but that it involves attempting particular conditions which were complied with by using only a particular type of producer microorganism for obtaining gold nanoparticles.

Practically all the processes involving producer microorganisms of substances or active ingredients comprise steps, which derive from promoting the conditions of and the appropriate culture medium so that said microorganisms develop and generate within the biomass the product of interest. Generally, in accordance with the present invention it has been found that biotechnological processes for the production of nanomaterials, to be carried out at levels or scales that can be considered industrial, require very importantly the identification and control of diverse variables so as to obtain the intended products which have diverse particularities which are not proper of all kinds of biotechnological processes.

Traditionally, the biotechnological products must be produced in cells grown in culture, which in practice allows to dominate a series of aspects not only of the research and development of production methods of these compounds but rather put into practise diverse strategies for scaling up processes and obtain the desired products. An important aspect is to mention the obtaining of biotechnological products such as the nanostructures or nanoparticles (different to proteins or any other traditional biotechnological product), implying that the bioprocesses in question must have an extremely particular performance.

In the regular practise, during the research and development phase, usually the initial production methods are developed at small scale. With all the research and development data derived from these production steps, production methods are developed at a large scale with the purpose of obtaining a sufficient quantity of the product for the intended market. The process of increasing scale and manufacturing will follow the same guidelines of good manufacturing practice to guarantee the safety and purity of the product. Nevertheless how routinely it may be, in traditional practice the process of increasing a cell culture to scale can be very difficult and requires a lot of time, and so, a lot of time is needed before a product can be obtained. The full process of production of a biotechnological product is usually divided into two main parts: manufacturing (upstream) and transformation (downstream). The manufacturing processes suppose the production of the biotechnological product, with higher frequency by using cells (from microbes, insects or mammals), which grow in culture. The transformation processes comprise the recovery, purification, formulation and conditioning of the product of interest following either standard procedures but each case of particular product, as are the magnetic nanoparticles (different from proteins or any other biotechnological product) require novel and better proposals for the transformation and set up of the nanostructures in the shape, size, features, quality, amount such, for their later uses.

In a little more detailed sense, the production phase starts with the cells or microorganism, which are created or designed for obtaining the protein product. In the case of the present invention, strains of microorganisms have been identified, prepared and conditioned specifically targeted for obtaining metallic magnetic nanoparticles, particularly gold nanoparticles, following a biotechnological process, which allows obtaining the nanostructures of interest in the intended quantity and quality to be used in different applications, as will be described later.

Once the desired cell line or producer microorganism has been obtained, it is subjected to cryopreservation or any other conservation means of the producer elements, so as to create a cells or microorganism bank. In the case of the production of magnetic nanoparticle producer microorganisms of the present invention, the selection, characterization and handling of the producer microorganisms allows obtaining nanoparticles with an improved process such that it matches or surpasses known chemical processes, maintaining the controlled growth of the producer microorganisms to generate in the biomass the quantity and quality of intended nanoparticles. Usually, the physical environment where the cell cultures or microorganisms are grown is monitored and controlled, so that in the case of the employees in the present invention it has been developed for the automatic control levels so that they allow a liable performance of this process stage, so that the nanoparticles are obtained at levels of bioreactors.

In the transformation phase of the manufacturing process, the product is isolated from the cells or microorganisms which produced it. The proteins present inside the cell (intracellular proteins) require some special protocols with the purpose of extracting them for their purification. Normally, this includes to suddenly opening the cells so as to release the protein product, which then must be purified with the remaining components that exist within the cell. The proteins present in the exterior of the cell (extracellular proteins) are usually easier to isolate with procedures, considered at this level of the state of the art, as traditional. This definitely does not happen when talking about products from the bioreactor in the form of a biomass containing magnetic nanoparticles obtained by the biological action of the producer microorganisms. The object method of separation and purification of the present invention assumes that it is of more complex products than simple proteins, which require improvements and substantially specific elements, and of inventive skill to achieve the separation of said products from the biomass generated in the bioreactor.

The economy of the biotechnological processes depends in great measure of the bioseparation operations involved, so that the correct selection of these operations has a strong impact on the success of the processes. The bioseparation processes involve the recovery and purification of products from the bioreactor. The bioseparations comprise all the treatments that the culture broth requires to obtain a biotechnological product in the purity and activity conditions required. It can be said that the success of a biotechnological process depends in gran measure of the suitable selection of the bioseparation process.

Usually, in general three generations of biotechnological processes can be distinguished, with respect to the type of bioseparations that these involve. The first generation comprises the group of processes developed by cultures of non-recombinant organisms, whose products are obtained in active form even if they are intracellular or if they are secreted to the culture medium. In this generation the traditional biotechnology processes as well as the production of ethanol, enzymes, citric acid, and antibiotics, are found. The products of these processes are presented in high concentrations at the start of the separation stage and do not require an extreme purity for their sale.

The discoveries associated with Molecular Biology and Genetic Engineering achieved particularly in the last decades allow the placement of a second generation of Biotechnology products such as human insulin, growth hormone, and alpha interferon, among others. These are produced intracellularly using Escherichia coli recombinant cells. They are characterised for being found at low concentrations within the cell, have a high molecular weight, have properties similar to contaminants and require a high grade of purity. Moreover, when produced in the cell they don't have biological activity because they are peptide chains without the appropriate conformation or structure; which is translated in the need to apply further physicochemical treatments to achieve the product in its active state.

The third biotechnology generation can be characterized by processes whereby extracellular products are obtained from recombinant cells or producer microorganisms, most of which are eukaryotic. In these systems the capacity not only to exogenously produce the desired product, but to obtain it in active form, such is the case of magnetic nanoparticles, which form part of the objective of the present invention, have been observed. The biotechnological processes of both the second and third generation constantly demand the knowledge of the physicochemical properties of the products and their contaminants, with the objective of selecting the suitable separation operations because of the high grade of purity required by the products. In the case of the process of the present invention, the magnetic nanoparticles are preferably prepared by an improved centrifugation process and mechanic lysis under very particular conditions to obtain nanoparticles in the intended conditions.

The aspects of yield and purity of the products are basic to determine the viability of a bioprocess, since, to achieve the grade of purification required by this type of products, the purification must be carried out in various steps, generally.

Once isolated and purified, the products are subjected to analysis protocols confirming by characterization, the presence and properties of the obtained products. In the case of the object of the present invention, the magnetic nanoparticles are characterized and assessed in a way that allows demonstrating the obtaining of these nanostructures showing the viability and effectiveness of the process up to superior objective scales of the present invention. After this step, the obtained nanoparticles are susceptible of being functionalised and used in different uses and applications.

Therefore, in accordance with the present invention the start point is producer microorganisms of the type representing bacteria type Brevibacterium halotolerans, Bacillus mojavensis, Bacillus subtilis subsp. Inaquosorum, Bacillus subtilis subsp. Spizizenii, Bacillus tequilensis, Bacillus amyloliquefaciens subsp. Amyloliquefaciens, Bacillus siamenensis, Bacillus amyloliquefaciens subsp. Plantarum, Bacillus subtilis, Bacillus subtilis subsp. Inaquosorum, Bacillus atrophaeus, or Bacillus vallismortis, from which in an “upstream” process step in the presence of a suitable culture medium and with the precursor material of the metallic element of interest, a biomass is obtained containing metallic magnetic nanoparticles, preferably of gold.

According to what is shown in FIG. 1, a block diagram of the objective process of the present invention is shown, in which the block (1) comprises the feeding of the nanoparticle producer microorganisms. These microorganisms according to the present invention comprise bacteria preferably of the type Brevibacterium halotolerans, Bacillus mojavensis, Bacillus subtilis subsp. Inaquosorum, Bacillus subtilis subsp. Spizizenii, Bacillus tequilensis, Bacillus amyloliquefaciens subsp. Amyloliquefaciens, Bacillus siamenensis, Bacillus amyloliquefaciens subsp. Plantarum, Bacillus subtilis, Bacillus subtilis subsp. Inaquosorum, Bacillus atrophaeus, or Bacillus vallismortis. More particularly, it refers to a bacteria of the type Bacillus mojavensis which have been identified by request of the applicant and carried out with the method consisting of: direct amplification by PCR of the 16S ARNr gene, partial sequencing thereof (with readings on both directions) and analysis of the sequences (Arahal et al., 2008) and deposited under the CO01 indication before the International Autority for the Deposit of Microorganisms named Colección Española de Cultivos Tipo (CECT), under the number CET 8698.

In the initial “upstream” stage, the growth step of the microorganism's in question inoculum is carried out in a liquid culture medium, using carbon and nitrogen sources. The percentage ratio thereof with respect to the total volume of fermentation will be able to stay in a preferred range of between 1 and 60%.

The second step is the preparation and sterilization of the culture medium for the fermentation (2), which is carried out dissolving in water the carbon sources, the nitrogen sources and salts of the metallic element of the magnetic nanoparticle of interest, which in the case of obtaining gold nanoparticles the use of gold chloride, gold chloride hydrate is preferred, or mixtures thereof.

Afterwards, the sterilization of the culture medium at a preferred temperature of between 85° C. and 150° C., preferably in the order of 115° C. and 125° C. and a pressure of between 5 and 25 pounds/inch² during a period of between 5 and 35 minutes, is carried out. Finally, the gold salts, such as, but not limited to gold chloride, gold chloride hydrate at a concentration of between 0.01 to 30 mM, are added to the cold culture medium, and it is put to stir at a speed such but not limited to 50, 80, 90, 100, 120, 140, 160 at a temperature such as but not determined as 15-20, 23, 27, 30, 33° C. during a period such but not limited to of 12, 24, 36, 48, 50 hours. Past the time, a final absorbance at 540 nm will be read to obtain the difference of growth between the initial and final absorbance.

According to the preferred embodiment of the present invention, the fermentation (3) is carried out by batch culture or fed batch culture, in stirred-tank like bioreactors with one or more impellers, such as, but not limited to a marine propel, flat pellets and/or Rushton type and tilted turbines, in controlled ranges (4) of temperature, pH, stirring and dissolved oxygen (OD) such as, but not limited to 10-55° C., 4-8, 30-500 rpm, and 0.1-1.5 vvm, respectively, during a period of time of from 5 to 200 hours.

In the stage called “downstream” after the fermentation, the first step is the centrifugation of the depleted broth (5) to concentrate the biomass. This is carried out at a speed of about 100 and 20 000 rpm, during a time of residence of between 1 and 180 minutes (6) in a discs centrifuge. Because the gold nanoparticles are produced intracellularly, the product must be extracted by mechanic lysis (7), which is carried out in devices such as, but not limited to, a ball grinder using pearls which can be of materials such as, but not limited to glass, metal and plastic under operation conditions (8) of turning speed and time of 100-2000 rpm and 1-120 minutes, respectively.

To separate the adhered cellular remains to the nanoparticles, these are sonicated (9) during a period, such as, but not limited to, of 5 to 240 minutes at a wavelength opening of between 1 and 90% (10). Afterwards, the culture medium is filtered (11) in at least three stages with the purpose of separating gold nanoparticles from the remaining components, as well as to generate define diameter ranges. For that, first, a microfiltration of the medium is carried out using a filtering medium with a pore diameter (12) such as, but not limited to, a range of between 0.22 and 5 μm; afterwards, a tangential ultrafiltration system is used, in which the medium is made to pass through a filter with a pore diameter such as, but not limited to of 0.1-0.06 μm. The obtained nanoparticles in the retainer (13) will be in the range of >100 nm, while those of the permeate are passed again through a filter of pore diameter such as, but not limited to, of 0.05 to 0.026 μm; the permeate can even be subjected to another filtration round through a filtering medium with a pore diameter such as, but not limited to, in a range of 0.025 μm to 10 kDa.

That is to say, once this stage is finished the gold nanoparticles will be obtained in the ranges such as, but not limited to, of 1 to 25 nm, 26 to 50 nm, 50 to 100 nm and >100 nm.

The final step is the formulation, which consists of the preparation of the intended gold nanoparticles for their final presentation, which can be an aqueous suspension with sodium citrate, citric acid or PBS or a powder presentation (14). If a suspension is desired, first the concentration is determined by UV-visible spectrophotometry and then it is taken to the required concentration obtaining the solution. If it is necessary to concentrate the nanoparticles, a centrifugation or lyophilisation can be carried out first. For the powder presentation, a lyophilisation at temperature and pressure ranges, such as but not limited to, from −80° C. to 20° C. and from 0.2 to 160 Pa, respectively, is carried out. Lastly, the gold nanoparticles are stored in closed containers for their final presentation.

To establish the above-described gold nanoparticles production process, it was important to first know the optimum conditions of preparation and growth of the microorganism, and the formulation of the production medium at laboratory level. Once these parameters were determined, the type of suitable culture medium for the production was established, as well as the purification method and finally, the formulation. According to this process, our invention is characterized for being suitable to operate processes for obtaining gold magnetic nanoparticles in volumes considered of scales superior to laboratory level, in reactors of the order of 7 to 10 litres.

To illustrate in more detail the above-described process and the results thereof put into practice, the following examples are provided as an explanation of the methods carried out to obtain the process for the biological production of the gold nanoparticles described in the present invention, as well as the methods for characterizing the obtained product.

### Examples

### Example 1: Method for Establishing the Optimum Production Conditions at Laboratory Level

The Bacillus mojavensis producer microorganism deposited under the indication CO01 before the International Authority for the Deposit of Microorganisms of name Colección Española de Cultivos Tipo (CECT), under the number CET 8698, was used, for which the optimum growth conditions and gold nanoparticles production rail, were established (upstream-fermentation-downstream) by an experimental design Box-Behnken (1960) for three factors (temperature, stirring speed and gold salts concentration) and three levels. That is, in total thirteen experiments in duplicate were carried out, of which 286 samples were obtained to which substrate, product and biomass concentrations were determined. Finally, thirteen more samples were further processed to determine the percentage of organic matter. Overall, a total of 1157 measurements were carried out.

The values for biomass and product were obtained with dry weight. The amount of substrate was determined by the 3,5-dinitrolsalicylic acid (DNS) technique described by Miller (1959), for which sucrose hydrolysis was previously carried out according to Godoy (2002) and ICONTEC (1994). The amount of organic matter was determined for each final sample of kinetics, by incineration (AOAC, 1990).

The separation of the nanoparticles was carried out in three stages: extraction, sonication and filtration. To carry out the wash, the samples were centrifuged at 3700 rpm for 20 minutes, the supernatant was decanted and the pellet was washed with distilled water, then it was centrifuged again under the same time and speed conditions. At the end, the liquid phase was removed.

For the extraction, two methods were tested: alkaline lysis and mechanical lysis. In the alkaline lysis the obtained cellular package was re-suspended in an equal proportion of volume of a NaOH solution at 20% and it was incubated at 37° C. for 4 hours. At the end of this time period, the cell suspension was subjected to sonication for 2 hours. The mechanic lysis was carried out adding an equal volume as glass pearls to the pellet and stirring in vortex for 5 minutes.

After carrying out the extraction, the samples were filtered, sonicated and serial-filtered with membranes of 0.22, 0.1, 0.05 and 0.025 μm. Afterwards, they were analysed with an electronic microscope (TEM and SEM).

The following table shows various samples that were handled according to the described process for obtaining gold nanoparticles.

TABLE 1
Box Behnken experimental design for the microorganism's kinetics

| | | Gold chloride | Sucrose |
|---|---|---|---|
| | | concentration | concentration in |
| Sample number | Temperature | (AuCl₃) in medium | medium |
| 1 | − | 0 | + |
| 2 | − | + | 0 |
| 3 | − | − | 0 |
| 4 | − | 0 | − |
| 5 | + | − | 0 |
| 6 | + | + | 0 |
| 7 | + | 0 | + |
| 8 | + | 0 | − |
| 9 | 0 | − | + |
| 10 | 0 | + | + |
| 11 | 0 | − | − |
| 12 | 0 | + | − |
| 13 | 0 | 0 | 0 |
| 14 | 0 | 0 | 0 |
| 15 | 0 | 0 | 0 |

TABLE 2
Independent variables and their variation levels.

| | Level | | | | | |
|---|---|---|---|---|---|---|
| Factor | − | | 0 | | + | |
| Temperature | 23° | C. | 28° | C. | 33° | C. |
| Gold chloride | 0.5 | mM | 1.5 | mM | 3 | mM |
| concentration in the | | | | | | |
| medium | | | | | | |
| Sucrose | 7.5 | g/L | 15 | g/L | 22.5 | g/L |
| concentration in the | | | | | | |
| medium | | | | | | |

Samples were taken every 1 or 2 hours according to the observed bacterial growth LP and the successive response variables were reported: growth specific rate (p); substrate consumption rate (qs); product generation rate (qp); and nanoparticle diameter.

The statistical analysis was carried out with support from MINITAB® 16 software; the obtained microorganism's kinetic parameters were: growth specific rate (p); gold nanoparticles performance with respect to an inductor (YPS); gold nanoparticles performance with respect to biomass (YPX); biomass performance with respect to a carbon source (YXS); Productivity (qP); and carbon source consumption-specific rate (qS).

The kinetic parameters were determined with the following methods: p.—dry weight before lysis; qp.—Dry weight-after lysis; qs.—3,5-dinitrosalicylic acid (DNS); Ypx.—Δp/Δx; Yxs.—consumed Δx/Δs; and Yps.—consumed Δp/Δs.

To calculate the growth specific rate the lineal form of the following formula was used:

$$\frac{dx}{dt} = μ x$$

wherein:

x=biomass concentration in g/L.

μ=growth specific rate in h−1.

To calculate the consumption specific rate of product generation, that is, of gold nanoparticles, (productivity) the following lineal formula was used:

$$\frac{dp}{dt} = {q}_{p} x$$

wherein:

x=biomass concentration in g/L.

qp=product generation specific rate in h−1.

To calculate the specific rate for substrate consumption the following lineal formula was used:

$$\frac{ds}{dt} = - {q}_{s} x$$

wherein:

x=biomass concentration in g/L.

qs=substrate consumption specific rate in h−1.

To determine the yields the following formulas were used:

$${Y}_{XS} = \frac{Δ X}{Δ S} [ = ] {g}_{biomass} \text{/} {g}_{sucrose}$$

$${Y}_{PC} = \frac{Δ P}{Δ C} [ = ] \frac{{g}_{AuNPts}}{{g}_{{AuCl}_{3}}}$$

$${Y}_{PX} = \frac{Δ P}{Δ X} [ = ] \frac{{g}_{AuNPts}}{{g}_{biomasa}}$$

wherein:

S=amount of consumed carbon source, expressed in g.

P=amount of AuNPs generated, expressed in g.

X=amount of generated biomass, expressed in g.

C=amount of gold salt consumed, expressed in g.

### Example 2. Method for Establishing the Optimum Production Conditions at Pilot Level

Once the optimum conditions for generating nanoparticles were determined, a batch culture in stirred tank-like bioreactors of 7 litres capacity, was established. As scaling up criteria, the geometric similarity and Reynolds Number were used, the latter because by maintaining the flow rate, the mixing, cut effort and air bubbles dispersion ensures that they are maintained constant, apart from favouring the micromixing.

In the case of the geometric similarity, the same ratios in the shape factors were maintained. Once the conditions at laboratory level, which gave the size and shape characteristics of the gold nanoparticles, were determined, fermentations were carried out in a stirring tank-like bioreactor, varying the conditions of stirring rate, pH and aeration, thereby obtaining the kinetic parameters (shown on Table 5) and the conditions with which nanoparticles with the desired sizes were obtained.

After specifying the fermentation process, the filtering process was established, and with that, the suspended organic matter from the product was eliminated and it was possible to separate the nanoparticles according to sizes.

Finally, various tests for the formulation were carried out, establishing the operating conditions of the drying equipment and the concentrations in the suspension with the purpose of obtaining a stable product.

TABLE 3
Box Behnken experimental design for the microorganism's kinetics

| | | Gold chloride | Sucrose |
|---|---|---|---|
| | | concentration | concentration in |
| Sample number | Temperature | (AuCl₃) in medium | medium |
| 1 | − | 0 | + |
| 2 | − | + | 0 |
| 3 | − | − | 0 |
| 4 | − | 0 | − |
| 5 | + | − | 0 |
| 6 | + | + | 0 |
| 7 | + | 0 | + |
| 8 | + | 0 | − |
| 9 | 0 | − | + |
| 10 | 0 | + | + |
| 11 | 0 | − | − |
| 12 | 0 | + | − |
| 13 | 0 | 0 | 0 |
| 14 | 0 | 0 | 0 |
| 15 | 0 | 0 | 0 |

TABLE 4
Independent variables and their variation levels.

| | Level | | | | | |
|---|---|---|---|---|---|---|
| Factor | − | | 0 | | + | |
| Temperature | 23° | C. | 28° | C. | 33° | C. |
| Gold chloride | 0.5 | mM | 1.5 | mM | 3 | mM |
| concentration in the | | | | | | |
| medium | | | | | | |
| Sucrose | 7.5 | g/L | 15 | g/L | 22.5 | g/L |
| concentration in the | | | | | | |
| medium | | | | | | |

Samples were taken every 1 or 2 hours according to the observed bacterial growth LP and the successive response variables were reported in the same manner of for the previous examples in terms of: growth specific rate (p); substrate consumption rate (qs); product generation rate (qp); and nanoparticle diameter.

Likewise, the statistical analysis was carried out with support from MINITAB® 16 software; the obtained microorganism's kinetic parameters were: growth specific rate (μ); gold nanoparticles performance with respect to an inductor (YPS); gold nanoparticles performance with respect to biomass (YPX); biomass performance with respect to a carbon source (YXS); Productivity (qP); and carbon source consumption-specific rate (qS).

Likewise, as in the previous Example 1, the kinetic parameters were determined; growth specific rate calculation, product generation consumption specific rate and substrate consumption specific rate.

In the graph appearing in FIG. 2 of the present invention, gold nanoparticles concentration and biomass and substrate concentrations with respect to time, all according to the product obtained as an embodiment described in this example, are shown.

The performance of determined parameters of the process for obtaining gold nanoparticles of the present invention with regards to Examples 1 and 2, at laboratory plant and pilot plant, respectively, were comparatively analysed.

TABLE 5
Kinetic parameters determined in a flask and a bioreactor.

| Parameter | Pilot plant reactor | Flask laboratory |
|---|---|---|
| μ (1/h) | 0.097 | 0.085 |
| qₚ (1/h) | 0.004 | 0.005 |
| qₛ (1/h) | 0.807 | 0.556 |
| Y_{PS} (g _{AuNPs}/g _{AuCl3}) | 0.534 | 0.54 |
| Y_{PX} (g A_{uNPs}/g _{biomass}) | 0.050 | 0.051 |
| Y_{XS} (g _{biomass}/g _{sucrose}) | 0.293 | 0.29 |

### Example 3. Gold Nanoparticles Characterization by UV-Visible Spectrophotometry

To characterize the gold nanoparticles by UV-Visible spectrophotometry, scanning at 400-800 nm wavelengths was carried out. The highest optical absorbance peak is shown in the ranges of 550-570 nm depending on the diameter. FIG. 3 shows spectra images to characterize the obtained gold nanoparticles, in which it is possible to acknowledge: (A) the established scan of a 400 to 600 nm wavelength; and (B) the established scan of a 400 to 800 nm wavelength.

### Example 4. Gold Nanoparticles Characterization by Transmission Electron Microscopy

A small sample of the obtained product according to the nanoparticles obtained in Example 2 was taken, after the filtrations, it was placed on a copper rack, which was placed under the transmission electron microscope brand JEOL model J1100, and was taken to 220 kV. The nanoparticles were observed and appear in FIGS. 4 and 9 of the present application, in which their size, shape and mono dispersion can be shown in more detail, thereby demonstrating that not only gold nanoparticles are obtained in quantity but in quality, important according to the spirit of the present invention.

Having described the invention in detail, it is considered as novel and so it is claimed as property what is contained in the following claims.

1. A biotechnological process for obtaining magnetic nanoparticles which comprises obtaining magnetic nanoparticles from producer microorganisms, wherein the process comprises the steps of:
selecting and preparing the producer microorganism;
growing said microorganism's inoculum;
sterilizing a culture medium;
adding a salt of a metallic element of interest;
fermenting the culture medium with the microorganism;
centrifugation of the depleted broth;
cell lysis to separate the obtained nanoparticles;
sonication of the cellular remains;
microfiltration and ultracentrifugation;
and final presentation of the obtained magnetic nanoparticles.

2. The biotechnological process for obtaining magnetic nanoparticles according to claim 1, wherein the producer microorganism is selected from the group of bacteria comprising: Brevibacterium halotolerans, Bacillus mojavensis, Bacillus subtilis subsp. Inaquosorum, Bacillus subtilis subsp. Spizizenii, Bacillus tequilensis, Bacillus amyloliquefaciens subsp. Amyloliquefaciens, Bacillus siamenensis, Bacillus amyloliquefaciens subsp. Plantarum, Bacillus subtilis, Bacillus subtilis subsp. Inaquosorum, Bacillus atrophaeus, or Bacillus vallismortis, amongst others.

3. The biotechnological process for obtaining magnetic nanoparticles according to claim 2, wherein the preferred producer microorganism is a bacteria of the type Bacillus mojavensis having the indication CO01 before the Colección Española de Cultivos Tipo (CECT) under the deposit number CET 8698.

4. The biotechnological process for obtaining magnetic nanoparticles according to claim 1, wherein the growth of said microorganism's inoculum is carried out in a liquid culture medium, using carbon and nitrogen sources in a percentage ratio with respect to total volume of fermentation of between 1 and 60%.

5. The biotechnological process for obtaining magnetic nanoparticles according to claim 1, wherein the step of preparing and sterilizing the culture medium for the fermentation is carried out by dissolving in water the carbon sources, the nitrogen sources and salts of the metallic element of the magnetic nanoparticle of interest.

6. The biotechnological process for obtaining magnetic nanoparticles according to claim 1, wherein the process is for obtaining gold nanoparticles, for which, the salt of the metallic element (gold) of interest used is gold chloride, gold chloride hydrated, or mixtures thereof.

7. The biotechnological process for obtaining magnetic nanoparticles according to claim 1, wherein the step of sterilization of the culture medium is carried out at a preferred temperature of between 85° C. and 150° C., preferably in the order or 115° C. and 125° C. and a pressure of between 5 and 25 pounds/inch² for a period of time of between 5 and 35 minutes.

8. The biotechnological process for obtaining magnetic nanoparticles according to claim 1, wherein the gold salt concentration in the culture medium is at a concentration of between 0.01 and 30 nM.

9. The biotechnological process for obtaining magnetic nanoparticles according to claim 1, wherein the reactor's operation conditions are of a stirring rate of between 50 and 160 rpm; a temperature of between 15 and 33° C. and a period of time of between 12 and 50 hours.

10. The biotechnological process for obtaining magnetic nanoparticles according to claim 1, wherein the reaction is carried out for a period of time until the final absorbance is in the order of 540 nm to obtain the difference of growth between the initial and final absorbance.

11. The biotechnological process for obtaining magnetic nanoparticles according to claim 1, wherein the fermentation is carried out: by batch culture or fed batch culture; in stirred tank-like bioreactors with one or more impellers, such as, but not limited to a marine propeller, flat pellets and/or Rushton and inclined type turbines; in controlled ranges of temperature of between 10 and 55° C.; at a pH of between 4 and 8; stirring rate of between 30 and 500 rpm; and dissolved oxygen (OD) of between 0.1-1.5 vvm; all in a period of time that can vary of between 5 and 200 hours.

12. The biotechnological process for obtaining magnetic nanoparticles according to claim 1, wherein the biomass or depleted fermentation broth is centrifuged to concentrate the biomass up to a rate of between 100 and 20,000 rpm, during a residence period of time of between 1 and 180 minutes, preferably in a discs centrifuge.

13. The biotechnological process for obtaining magnetic nanoparticles according to claim 1, wherein the intracellularly obtained nanoparticles are extracted by mechanic lysis, using devices such as glass, metal and plastic balls grinder, at rates of between 100 to 2,000 rpm and during a period of time of 1 to 120 minutes.

14. The biotechnological process for obtaining magnetic nanoparticles according to claim 13, wherein the cellular remains adhered to the nanoparticles are sonicated during a preferred period of time of between 5 and 240 minutes at a wavelength of between 1 and 90%.

15. The biotechnological process for obtaining magnetic nanoparticles according to claim 1, wherein the obtaining of the nanoparticles is carried out by filtering with a pore size of between 0.22 to 5 μm, followed by a tangential ultrafiltration system with various filters of pore diameter of between 0.1 to 0.6 μm, 0.05 to 0.26 μm and of 0.025 to 10 kDa.

16. The biotechnological process for obtaining magnetic nanoparticles according to claim 1, wherein the obtained nanoparticles in the first retainer are bigger than 100 nm, while those of the permeate are inferior to 100 nm.

17. The biotechnological process for obtaining magnetic nanoparticles according to claim 1, wherein stable and mono dispersed gold magnetic nanoparticles are obtained of between 1 and 100 nm, which can be presented in an aqueous suspension of sodium citrate, citric acid or PBS or in a powder.
